(19) United States
(12) Patent Application Publication
(10) Pub. No.: US 2024/0170114 A1
(43) Pub. Date: May 23, 2024

(54) SYSTEMS AND METHODS FOR INCREASING THERMOMETRIC RELIABILITY

(71) Applicant: Helen of Troy Limited, St. Michael (BB)

(72) Inventors: Kevin M. Johnson, Natick, MA (US); John D. Gundlach, Auburndale, MA (US)

(21) Appl. No.: 18/552,790

(22) PCT Filed: Mar. 22, 2022
(86) PCT No.: PCT/US2022/021298

§ 371 Date: Sep. 27, 2023

Related U.S. Application Data
(60) Provisional application No. 63/167,848, filed on Mar. 30, 2021.

(51) Int. Cl. G16H 10/60; G16H 10/40

(52) U.S. Cl. CPC G16H 10/60; G16H 10/40

(57) ABSTRACT

Systems and methods for increasing thermometric reliability are provided. In one embodiment, a system includes a temperature module, an analysis module, and a recommendation module. The temperature module is configured to receive an inquiry from the user about a current measured temperature. The current measured temperature is received from a thermometer. The inquiry indicates that the user doubts the current measured temperature. The analysis module is configured to generate a query based on the inquiry and the current measured temperature. The analysis module is also configured to receive an answer in response to the query. The analysis module is further configured to analyze the current measured temperature in an analysis of historical data. The recommendation module is configured to generate a recommendation associated with the inquiry based on the answer and the analysis.

### BACKGROUND

The mercury thermometer was the standard for measuring body temperature for decades. Commonly, a mercury thermometer was formed with a hollow glass cylinder having a bulb with mercury at one end and being closed at the other end. To measure a user's temperature, the bulb end was inserted into the user, for example, under the user's tongue. In addition to being uncomfortable and awkward, the placement of the mercury thermometer could be dangerous as mercury is poisonous to humans; should the cylinder or rod break, the user may inadvertently ingest the mercury.

More recently, electronic thermometers have become widely available. Electronic thermometers may measure the body temperature in a myriad of ways based on the sensors included in the electronic thermometer. For example, an electronic thermometer may have a temperature sensing tip at one end for insertion, for example, under the tongue of the user. Alternatively, an electronic thermometer may be set against the temple of a user and swiped along their forehead. An electronic thermometer may also be contactless such that it is aimed at a user and triggered to measure the body temperature of the user. Based on the way that the electronic thermometer measures temperature, a number of factors may influence a reading, including position of the electronic thermometer, environment, etc. Therefore, a user may be unsure whether the body temperature has been correctly measured based on sensors of the electronic thermometer or whether the measured body temperature was influenced by another factor. Accordingly, a user may begin to distrust the measured body temperatures of an electronic thermometer despite sensors of electronic thermometers typically being more accurate than a mercury thermometer.

### SUMMARY

According to one embodiment, a system for increasing thermometric reliability is provided. The system includes a temperature module, an analysis module, and a recommendation module. The temperature module is configured to receive an inquiry from the user about a current measured temperature. The current measured temperature is received from a thermometer. The inquiry indicates that the user doubts the current measured temperature. The analysis module is configured to generate a query based on the inquiry and the current measured temperature. The analysis module is also configured to receive an answer in response to the query. The analysis module is further configured to analyze the current measured temperature in an analysis of historical data. The recommendation module is configured to generate a recommendation associated with the inquiry based on the answer and the analysis.

In the system, the historical data may include previously measured temperatures for the user that precede the current measured temperature by a predetermined amount of time that defines a session. The previously measured temperatures for the user may be associated with a profile for the user. The analysis may include performing a trend line analysis of the historical data and the current measured temperature. The analysis of the system may include determining a variability value for the historical data and generating the query to confirm the profile of the user in response to variability value exceeding a variability threshold. The profile may also include location data associated with the user and/or demographic information about the user. The path that the query takes can be affected by one or more of these factors, enabling the user to achieve resolution and a more accurate temperature as quickly as possible.

In some embodiments of the system, the query may request verification of the profile. Additionally or alternatively, the profile may also include a first set of the historical data associated with a first anatomical region and a second set of the historical data associated with a second anatomical region different than the first anatomical region. The query may the user specify whether the current measured temperature was measures at the first anatomical region or the second anatomical region. The recommendation of the system may further be based on the first set of the historical data or the second set of the historical data based on the answer. The recommendation may include manipulating the thermometer.

According to another embodiment, a method for increasing thermometric reliability is provided. The method includes receiving an inquiry from the user about a current measured temperature. The current measured temperature is received from a thermometer. The inquiry indicates that the user doubts the current measured temperature. The method includes generating a query based on the inquiry and the current measured temperature. The method further includes receiving an answer in response to the query. The method yet further includes analyzing the current measured temperature in an analysis of historical data. The method includes generating a recommendation associated with the inquiry based on the answer and the analysis.

The historical data of the method may include previously measured temperatures for the user that precede the current measured temperature by a predetermined amount of time that defines a “Session.” The previously measured temperatures for the user may be associated with a profile for the user. The analysis may include performing a trend line analysis of the historical data and the current measured temperature. The analysis may include determining a variability value for the historical data and generating the query to confirm the profile of the user in response to variability value exceeding a variability threshold. The profile may also include location data and/or demographic information about the user.

According to yet another embodiment, a non-transitory computer readable storage medium storing instructions that, when executed by a computer having a processor, cause the computer to perform a method for increasing thermometric reliability. The method includes receiving an inquiry from the user about a current measured temperature. The current measured temperature is received from a thermometer. The inquiry indicates that the user doubts the current measured temperature. The method includes generating a query based on the inquiry and the current measured temperature. The method further includes receiving an answer in response to the query. The method yet further includes analyzing the current measured temperature in an analysis of historical data. The method includes generating a recommendation associated with the inquiry based on the answer and the analysis.

### BRIEF DESCRIPTION OF THE DRAWINGS

FIG. 1 is an exemplary operating environment of a system for increasing thermometric reliability, according to one aspect.

FIG. 2 is an exemplary user embodiment for a system for increasing thermometric reliability, according to one aspect.

FIG. 3 is an exemplary process flow of a method for increasing thermometric reliability, according to one aspect.

FIG. 4 is an exemplary framework for increasing thermometric reliability, according to one aspect.

FIG. 5 is an exemplary portable device embodiment for a system for increasing thermometric reliability, according to one aspect.

FIG. 6 is an exemplary process flow for session limiting in analysis for increasing thermometric reliability, according to one aspect.

FIG. 7 is an exemplary process flow for an iterative analysis for increasing thermometric reliability, according to one aspect.

FIG. 8 is an exemplary process flow for generating a query with a number of requests associated with increasing thermometric reliability according to one aspect.

FIG. 9 is an exemplary process flow for providing an estimated temperature to increase thermometric reliability, according to one aspect.

FIG. 10 is an illustration of an example computer-readable medium or computer-readable device including processor-executable instructions configured to embody one or more of the provisions set forth herein, according to one aspect.

### DETAILED DESCRIPTION

A user may not trust a measured temperature from an electronic thermometer electronic thermometer when the user gets a temperature reading that the user does not expect. Instead, the user is left wondering whether the user themselves made an error or whether the unexpected temperature reading is based on the thermometer working improperly. Additionally, or alternatively, the user may be concerned about external factors that affect the accuracy of the thermometer. The user may also just misunderstand a “good measurement.”

To give the user confidence in the measured temperature from the electronic thermometer, the systems and methods described herein simplify the complicated set of potential contributing factors for the user. Generally, the systems and methods described herein, analyze the measured temperature in light of historical data associated with the thermometer and/or the answers that the user provides in response to questions. The user may then be offered guidance in the form of a recommendation. Because the recommendation is based on the historical data and user answers, the user is provided with relevant, prioritized, and personalized guidance, thereby, improving the user's experience by reducing confusion and uncertainty.

### Definitions

The following includes definitions of selected terms employed herein. The definitions include various examples and/or forms of components that fall within the scope of a term and that may be used for implementation. The examples are not intended to be limiting. Furthermore, the components discussed herein, may be combined, omitted, or organized with other components or into different architectures.

“Bus,” as used herein, refers to an interconnected architecture that is operably connected to other computer components inside a computer or between computers. The bus may transfer data between the computer components. The bus may be a memory bus, a memory processor, a peripheral bus, an external bus, a crossbar switch, and/or a local bus, among others. The bus may also interconnect with components inside a device using protocols such as Media Oriented Systems Transport (MOST), Controller Area network (CAN), Local Interconnect network (LIN), among others.

“Component,” as used herein, refers to a computer-related entity (e.g., hardware, firmware, instructions in execution, combinations thereof). Computer components may include, for example, a process running on a processor, a processor, an object, an executable, a thread of execution, and a computer. A computer component(s) may reside within a process and/or thread. A computer component may be localized on one computer and/or may be distributed between multiple computers.

“Computer communication,” as used herein, refers to a communication between two or more communicating devices (e.g., computer, personal digital assistant, cellular telephone, network device, vehicle, connected thermometer, infrastructure device, roadside equipment) and may be, for example, a network transfer, a data transfer, a file transfer, an applet transfer, an email, a hypertext transfer protocol (HTTP) transfer, and so on. A computer communication may occur across any type of wired or wireless system and/or network having any type of configuration, for example, a local area network (LAN), a personal area network (PAN), a wireless personal area network (WPAN), a wireless network (WAN), a wide area network (WAN), a metropolitan area network (MAN), a virtual private network (VPN), a cellular network, a token ring network, a point-to-point network, an ad hoc network, a mobile ad hoc network, a vehicular ad hoc network (VANET), among others.

Computer communication may utilize any type of wired, wireless, or network communication protocol including, but not limited to, Ethernet (e.g., IEEE 802.3), WiFi (e.g., IEEE 802.11), communications access for land mobiles (CALM), WiMax, Bluetooth, Zigbee, ultra-wideband (UWAB), multiple-input and multiple-output (MIMO), telecommunications and/or cellular network communication (e.g., SMS, MMS, 3G, 4G, LTE, 5G, GSM, CDMA, WAVE, CAT-M, LoRa), satellite, dedicated short range communication (DSRC), among others.

“Communication interface” as used herein may include input and/or output devices for receiving input and/or devices for outputting data. The input and/or output may be for controlling different features, components, and systems. Specifically, the term “input device” includes, but is not limited to: keyboard, microphones, pointing and selection devices, cameras, imaging devices, video cards, displays, push buttons, rotary knobs, and the like. The term “input device” additionally includes graphical input controls that take place within a user interface which may be displayed by various types of mechanisms such as software and hardware-based controls, interfaces, touch screens, touch pads or plug and play devices. An “output device” includes, but is not limited to, display devices, and other devices for outputting information and functions.

“Computer-readable medium,” as used herein, refers to a non-transitory medium that stores instructions and/or data. A computer-readable medium may take forms, including, but not limited to, non-volatile media, and volatile media. Non-volatile media may include, for example, optical disks, magnetic disks, and so on. Volatile media may include, for example, semiconductor memories, dynamic memory, and so on. Common forms of a computer-readable medium may include, but are not limited to, a floppy disk, a flexible disk, a hard disk, a magnetic tape, other magnetic medium, an ASIC, a CD, other optical medium, a RAM, a ROM, a memory chip or card, a memory stick, and other media from which a computer, a processor or other electronic device may read.

“Database,” as used herein, is used to refer to a table. In other examples, “database” may be used to refer to a set of tables. In still other examples, “database” may refer to a set of data stores and methods for accessing and/or manipulating those data stores. In one embodiment, a database may be stored, for example, at a disk, data store, and/or a memory. A database may be stored locally or remotely and accessed via a network.

“Data store,” as used herein may be, for example, a magnetic disk drive, a solid-state disk drive, a floppy disk drive, a tape drive, a Zip drive, a flash memory card, and/or a memory stick. Furthermore, the disk may be a CD-ROM (compact disk ROM), a CD recordable drive (CD-R drive), a CD rewritable drive (CD-RW drive), and/or a digital video ROM drive (DVD ROM). The disk may store an operating system that controls or allocates resources of a computing device.

“Display,” as used herein may include, but is not limited to, LED display panels, LCD display panels, CRT display, touch screen displays, among others, that often display information. The display may receive input (e.g., touch input, keyboard input, input from various other input devices, etc.) from a user. The display may be accessible through various devices, for example, though a remote system. The display may also be physically located on a portable device or mobility device.

“Logic circuitry,” as used herein, includes, but is not limited to, hardware, firmware, a non-transitory computer readable medium that stores instructions, instructions in execution on a machine, and/or to cause (e.g., execute) an action(s) from another logic circuitry, module, method and/or system. Logic circuitry may include and/or be a part of a processor controlled by an algorithm, a discrete logic (e.g., ASIC), an analog circuit, a digital circuit, a programmed logic device, a memory device containing instructions, and so on. Logic may include one or more gates, combinations of gates, or other circuit components. Where multiple logics are described, it may be possible to incorporate the multiple logics into one physical logic. Similarly, where a single logic is described, it may be possible to distribute that single logic between multiple physical logics.

“Memory,” as used herein may include volatile memory and/or nonvolatile memory. Non-volatile memory may include, for example, ROM (read only memory), PROM (programmable read only memory), EPROM (erasable PROM), and EEPROM (electrically erasable PROM). Volatile memory may include, for example, RAM (random access memory), synchronous RAM (SRAM), dynamic RAM (DRAM), synchronous DRAM (SDRAM), double data rate SDRAM (DDRSDRAM), and direct RAM bus RAM (DRRAM). The memory may store an operating system that controls or allocates resources of a computing device.

“Module,” as used herein, includes, but is not limited to, non-transitory computer readable medium that stores instructions, instructions in execution on a machine, hardware, firmware, software in execution on a machine, and/or combinations of each to perform a function(s) or an action(s), and/or to cause a function or action from another module, method, and/or system. A module may also include logic, a software-controlled microprocessor, a discrete logic circuit, an analog circuit, a digital circuit, a programmed logic device, a memory device containing executing instructions, logic gates, a combination of gates, and/or other circuit components. Multiple modules may be combined into one module and single modules may be distributed among multiple modules.

“Operable connection,” or a connection by which entities are “operably connected,” is one in which signals, physical communications, and/or logical communications may be sent and/or received. An operable connection may include a wireless interface, firmware interface, a physical interface, a data interface, and/or an electrical interface.

“Portable device,” as used herein, is a computing device typically having a display screen with user input (e.g., touch, keyboard) and a processor for computing. Portable devices include, but are not limited to, handheld devices, mobile devices, smart phones, laptops, tablets, e-readers, smart speakers. In some embodiments, a “portable device” could refer to a remote device that includes a processor for computing and/or a communication interface for receiving and transmitting data remotely.

“Processor,” as used herein, processes signals and performs general computing and arithmetic functions. Signals processed by the processor may include digital signals, data signals, computer instructions, processor instructions, messages, a bit, a bit stream, that may be received, transmitted and/or detected. Generally, the processor may be a variety of various processors including multiple single and multicore processors and co-processors and other multiple single and multicore processor and co-processor architectures. The processor may include logic circuitry to execute actions and/or algorithms. The processor may also include any number of modules for performing instructions, tasks, or executables.

“User” as used herein may be a biological being, such as humans (e.g., adults, children, infants, etc.).

A “wearable computing device,” as used herein can include, but is not limited to, a computing device component (e.g., a processor) with circuitry that can be worn or attached to user. In other words, a wearable computing device is a computer that is subsumed into the personal space of a user. Wearable computing devices can include a display and can include various sensors for sensing and determining various parameters of a user. For example, location, motion, and physiological parameters, among others. Exemplary wearable computing devices can include, but are not limited to, watches, glasses, clothing, gloves, hats, shirts, jewelry, rings, earrings necklaces, armbands, leashes, collars, shoes, earbuds, headphones and personal wellness devices.

### System Overview

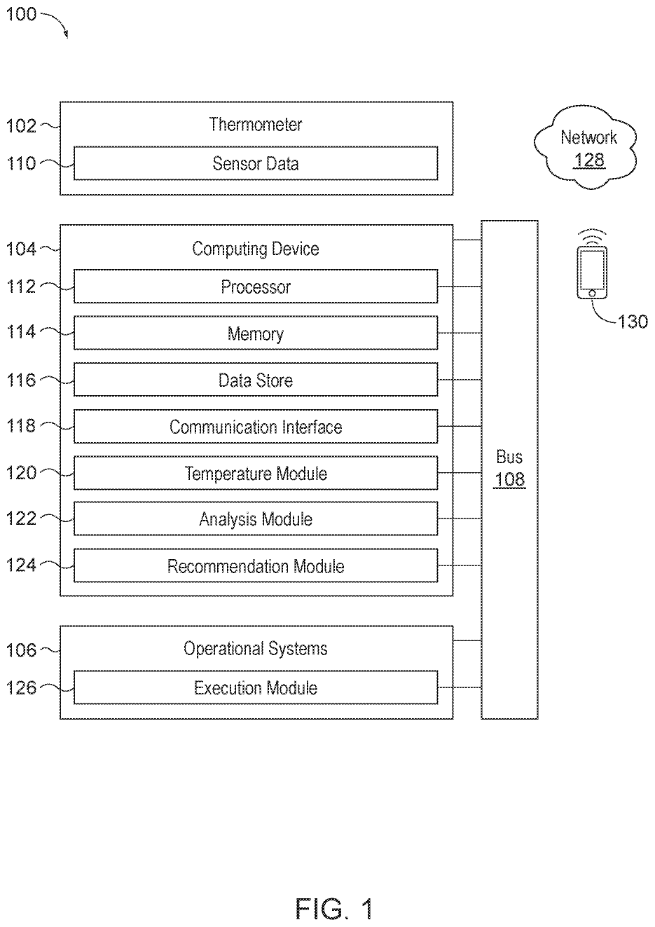

Referring now to the drawings, the drawings are for purposes of illustrating one or more exemplary embodiments and not for purposes of limiting the same. FIG. 1 is an exemplary component diagram of an operating environment 100 for increasing thermometric reliability, according to one aspect. The operating environment 100 includes a thermometer 102, a computing device 104, and operational systems 106. The thermometer 102, the computing device 104, and the operational systems 106 may be interconnected by a bus 108. The components of the operating environment 100, as well as the components of other systems, hardware architectures, and software architectures discussed herein, may be combined, omitted, or organized into different architectures for various embodiments. The computing device 104 may be implemented with a device or remotely stored.

The computing device 104 may be implemented as a part of a thermometer 102. The computing device 104 may be implemented as part of a telematics unit, a head unit, or an electronic control unit, among other potential systems of the thermometer 102. In other embodiments, the components and functions of the computing device 104 can be implemented with other devices such as portable device 130, database, remote server, or another device connected via a network (e.g., a network 128).

The computing device 104 may be capable of providing wired or wireless computer communications utilizing various protocols to send and receive electronic signals internally to and from components of the operating environment 100. Additionally, the computing device 104 may be operably connected for internal computer communication via the bus 108 (e.g., a Controller Area Network (CAN) or a Local Interconnect Network (LIN) protocol bus) to facilitate data input and output between the computing device 104 and the components of the operating environment 100.

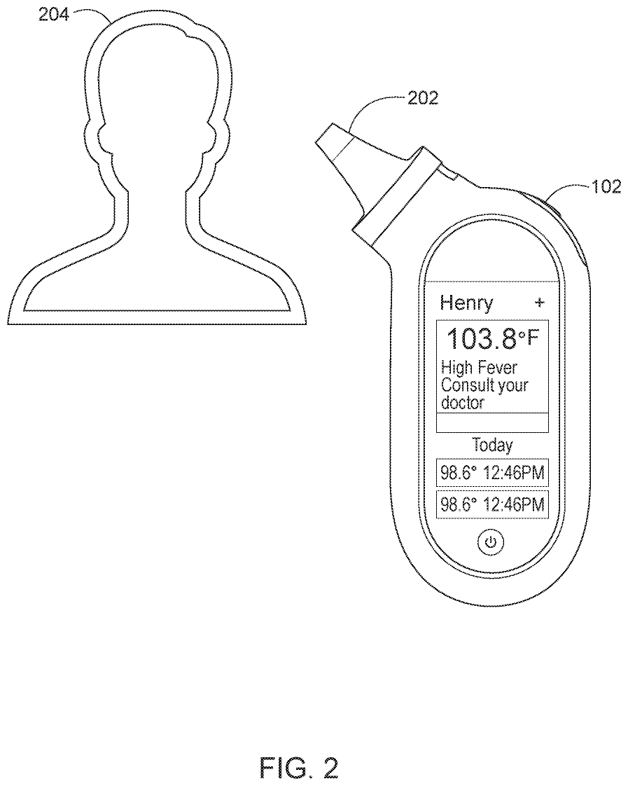

The thermometer 102 includes sensors for measuring temperature. For example, the thermometer 102 may include sensors for sensing the body temperature of a user 204. The sensors may include infrared sensors, thermistors, semiconductor-based temperature sensors, etc. Sensors of the thermometer 102 may be positioned on the thermometer 102 based on the type of the thermometer 102. For example, suppose that the thermometer 102 is a tympanic thermometer. The thermometer 102 may include a probe 202, shown in FIG. 2, that includes an infrared sensor (not shown) to measure the temperature inside the ear canal of a user 204. The thermometer 102 receives the measured temperature as sensor data 110. Accordingly, the sensors and/or the thermometer 102 are operable to sense a measurement of sensor data 110 associated with the user and generate a data signal indicating said measurement of data. The sensor data 110 may also be converted into other data formats (e.g., numerical) and/or used by the thermometer 102, the computing device 104, and/or the operational systems 106 to generate new sensor data 110 including data metrics and parameters. The sensor data 110 includes measured temperatures from the thermometer 102. The sensor data 110 may further include temperature data from the environment such as the ambient environmental data. For example, the thermometer 102 may include a sensor for measuring the temperature of the environment of the thermometer 102.

The computing device 104 includes a processor 112, a memory 114, a data store 116, and a communication interface 118, which are each operably connected for computer communication via a bus 108 and/or other wired and wireless technologies. The communication interface 118 provides software and hardware to facilitate data input and output between the components of the computing device 104 and other components, networks, and data sources, which will be described herein. Additionally, the computing device 104 also includes a temperature module 120, an analysis module 122, and a recommendation module 124, for increasing thermometric reliability facilitated by the components of the operating environment 100.

The computing device 104 is also operably connected for computer communication (e.g., via the bus 108 and/or the communication interface 118) to one or more operational systems 106. The operational systems 106 can include, but are not limited to, any automatic or manual systems that can be used to enhance the thermometer 102, operation by the user, and/or safety. The operational systems 106 include an execution module 126. The execution module 126 monitors, analyzes, and/or operates the thermometer 102, to some degree. For example, the execution module 126 may store, calculate, and provide information about the thermometer 102, such as previous usage statistics. The operational systems 106 also include and/or are operably connected for computer communication to the thermometer 102. For example, one or more sensors of the thermometer 102 may be incorporated with execution module 126 to monitor characteristics of the thermometer 102 such as location, position of the thermometer 102, etc. In another embodiment, the thermometer 102 may communicate with one or more devices or services (e.g., a wearable device, non-wearable device, cloud service, etc.) to monitor characteristics of the environment, such as the outdoor temperature given the location of the user 204. For example, the thermometer may the local temperature when a temperature measurement of the user 204 is measured.

The thermometer 102, the computing device 104, and/or the operational systems 106 are also operatively connected for computer communication to and via the network 128. The network 128 is, for example, a data network, the Internet, a wide area network (WAN) or a local area (LAN) network. The network 128 serves as a communication medium to various remote devices (e.g., databases, web servers, remote servers, application servers, intermediary servers, client machines, or other portable devices).

The operating environment 100 facilitates improving a user's experience by increasing thermometric reliability by interacting with the user 204 in order to provide the user 204 with context for a measured temperature. Furthermore, the systems and methods provide recommendations regarding the measured temperature so that the user 204 is confident in temperatures measured by the thermometer 102. Detailed embodiments describing exemplary methods using the system and network configuration discussed above will now be discussed in detail.

### II. Methods for Increasing Thermometric Reliability

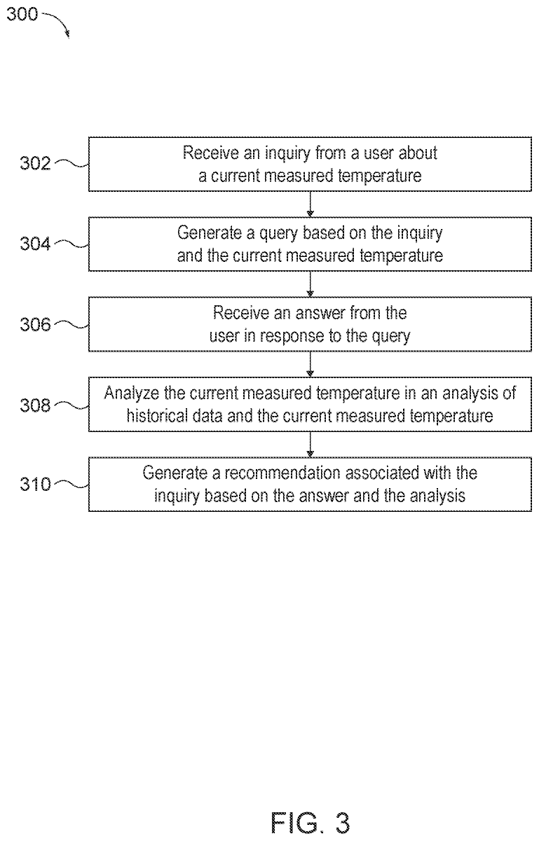

Referring to FIG. 3, a method 300 for increasing thermometric reliability will be described according to an exemplary embodiment. FIG. 3 will be described with reference to FIGS. 1, 2, 4, and 5. For simplicity, the method 300 will be described as a sequence of blocks, but the elements of the method 300 can be organized into different architectures, elements, stages, and/or processes.

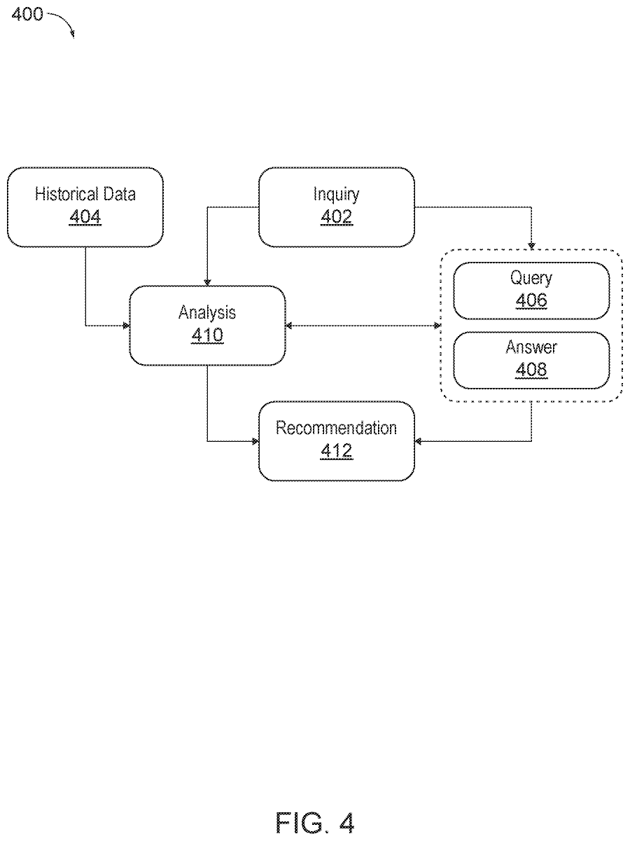

At block 302, the method 300 includes the temperature module 120 receiving an inquiry 402 from a user 204 about a current measured temperature for a subject. The temperature module 120 may receive the current measured temperature from thermometer 102 via the network 128, the portable device 130, or other form of computer communication. The inquiry 402 shown in the diagram 400 of FIG. 4 indicates that the user 204 doubts the current measured temperature.

The user 204 receives the current temperature measurement which is a measurement of the body temperature of the subject. The current temperature measurement may be received as the sensor data 110 from the thermometer 102. In some embodiments, the user 204 and the subject are distinct entities. For example, the user 204 may be a caregiver and the subject may be the charge of the caregiver. In other embodiments, the user 204 may be the subject. For example, the user 204 may be measuring his or her own body temperature.

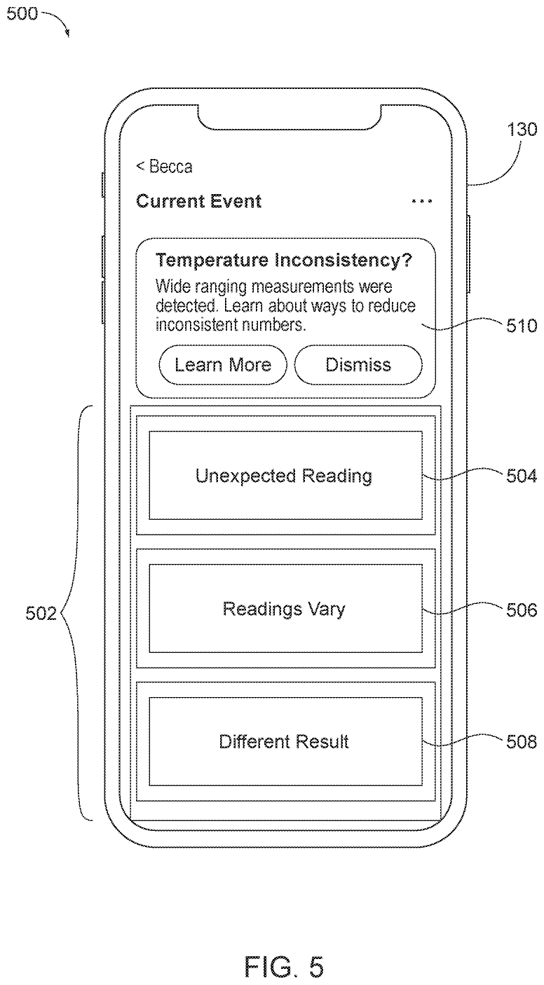

Suppose the user 204 is taking his or her own temperature and is thus, both the subject and the user 204. The user 204 may receive the current measured temperature on a display of the thermometer 102, as shown in FIG. 2, or on a display 500 of the portable device 130 shown in FIG. 5. If the user 204 is skeptical of the current measured temperature, the user 204 may use the thermometer 102 or the portable device 130 to input the inquiry 402. The user 204 may input the inquiry 402 as a free word input in which the user 204 provides keywords. In another embodiment, the temperature module 120 may provide the user 204 a set of inquiries 502 from which the user selects, and the selected inquiry is then received by the temperature module 120 as the inquiry 402 from the user 204.

Turning to FIG. 5, the set of inquiries 502 may include a number of inquiry categories that describe reasons that a user 204 would be skeptical of the current measured temperature of the thermometer 102. For example, suppose the user 204 is taking a routine temperature measurement required for entry to an establishment and the user 204 feels fine. The user 204 may be expecting to get their normal temperate as their current measured temperature, for example, approximately 98.6° F. If instead, the user 204 receives a current measured temperature of 104.1° F., the user 204 may be skeptical because the user 204 suspects that the current measured temperature is too high. Alternatively, if the user 204 is feeling poorly and flushed, the user 204 may expect to receive a higher temperature, for example, in excess of 100° F. If instead, the user 204 receives a current measured temperature of 97.3° F., the user 204 may be skeptical because the user 204 suspects that the current measured temperature is too low. Accordingly, one inquiry category of the of the set of inquiries 502 may be an unexpected reading category 504 that the user 204 may select if the current measured temperature is too high or too low relative to the user's expected reading.

Turning now to an example in which the user 204 and the subject are distinct. Suppose that the user 204 is a mother and the subject is a child. The user 204 may expect that the subject will have a different temperature. Suppose the user 204 observes that the child is weak, shivering, and sweating and expects that the child has a fever. However, the current measured temperature received from the thermometer 102 is 98.6° F. The mother as the user 204 may select the “unexpected reading” category 504 to indicate that the mother suspects that the current measured temperature is too low.

Another inquiry category of the set of inquiries 502 may include a “readings vary” category 506. The readings vary category 506 may be selected by the user 204 when the user 204 takes a number of temperature readings that fluctuate. For example, suppose that the user 204 measures three temperatures. The three temperatures may include a first previously measured temperature of 97.3° F., a second previously measured temperature of 102.5° F., and the current measured temperature of 98.6° F. The user 204 may not have confidence in the current measured temperature because of the irregular rising of the temperature from the first previously measured temperature to the second previously measured temperature. Additionally or alternatively, the user 204 may not have confidence in the current measured temperature because of the irregular falling of the temperature from the second previously measured temperature to the current measured temperature. The user 204 may also find the fluctuating between the three temperatures disconcerting. Based on any or all of these scenarios with respect to the three temperatures, the user 204 may select the readings vary category 506 on the display 500 of the portable device 130.

The temperature module 120 may also provide a different result category 508 in the set of inquiries 502. The different result category 508 may be selected by the user 204 if the user 204 is using multiple devices to sense the current measured temperature. For example, suppose that a user 204 measures his or her temperature using an alternative thermometer (not shown). If the user 204 then receives a different temperature reading from the thermometer 102, as the current measured temperature. If the previously measured temperature, received from the alternative thermometer, and the current measured temperature, received from the thermometer 102, are different, then the user 204 may select the different result category 508 to indicate that the user 204 is unsure of which device, the thermometer 102 or the alternative thermometer, gave a more accurate reading.

The inquiry categories are merely exemplary, and more, fewer, and/or different inquiry categories may be included in the set of inquiries 502. The temperature module 120 may receive the inquiry 402 from the user 204 as a selection of the inquiry category on the thermometer 102 or the display 500 of the portable device 130. Moreover, the set of inquiries 502 may be displayed in any configuration. For example, the temperature module 120 may receive a selection of an inquiry category based on the interaction with the display 500 or as an audio input. In one embodiment, the user 204 may respond to the temperature module 120 providing the set of inquiries 502 by audibly selecting an inquiry category. For example, the user 204 may say “I expected a higher temperature” or “I expected a lower reading.” The temperature module 120 may process either statement as a selection of the unexpected reading category 504. Likewise, the temperature module 120 may process the statement, “my other thermometer gave me a different result” as different result category 508.

In one embodiment, the temperature module 120 may present the set of inquiries 502 in response to input from the user 204. For example, the user 204 may express “I got a temperature I didn't expect,” either as an audible statement or through a manual entry, for example, on the display 500 of the portable device 130. As another embodiment, the temperature module 120 may present the set of inquiries 502 based on historical data 404 associated with the user 204.

The historical data 404 may include measured temperatures associated with the subject, regardless of whether the subject is a distinct entity or the user 204. This historical data 404 may include the sensor data 110 such as timestamps and/or a timeline for the measured temperatures. For example, the temperature module 120 may apply timestamps to temperature measurements received from the thermometer. Returning to the example given above in which three temperatures are received, the historical data 404 includes the first previously measured temperature, the second previously measured temperature, and the current measured temperature for the subject. Accordingly, measurements received for a subject may be stored in the memory 114 or data store 116 and accessed later by the temperature module 120. In another embodiment, the temperature module 120 may access the historical data 404 on a remote server (not shown) or database (not shown) accessible by the network 128.

The historical data 404 may be saved with respect to a profile for the subject or the user 204. The profile may include location data (e.g., position data, coordinates, weather information for the location, ambient environmental data, etc.) associated with the subject, demographic information (e.g., age, gender, ethnicity, etc.) and health information (e.g., health status, chronic conditions, current medications, temperature baseline, etc.) about the subject, as well as the historical data 404. The profile may be maintained by the subject, the user 204, or a third party. Suppose that the user 204 is a nurse that is measuring the body temperature of the subject, a patient. The nurse may not be confident in the current temperature measurement of the patient. The profile, including the historical data 404, is not associated with the person operating the thermometer 102, here the user 204, but rather the subject. For example, a hospital may maintain patient records that include historical data 404 about the subject. In this manner, the user 204 receives the current temperature measurement of the subject. In embodiments, in which the user 204 is measuring the body temperature of his or herself, the profile is associated with the user 204 because the user 204 is the subject.

Suppose that the user 204 receives the current measured temperature of the subject, but that relative to the historical data 404, the current measured temperature is inconsistent. The portable device 130 may display an inconsistency notification 510 to alert the user 204 that various measurements were detected for the subject. The inconsistency notification 510 may be displayed on the display 500 and be selectable by the user 204 as the inquiry 402. Accordingly, if selected, the inconsistency notification 510 may be received by the temperature module 120 as the inquiry 402 from a user 204 about a current measured temperature for a subject. The current measured temperature of the subject may be the most recent measured temperature of the historical data 404.

To avoid an inconsistency notification 510 from being displayed, when different measuring devices are being used or different anatomical regions of the subject are being measured, the historical data 404 may be chunked into temperature measurements based on measurement characteristics. The measurement characteristics may include the type of measurement, device used, anatomical area measured, among others. For example, the profile of the subject may include a first set of the historical data 404 associated with a first anatomical region of the subject and a second set of the historical data 404 associated with a second anatomical region of the subject that is different than the first anatomical region.

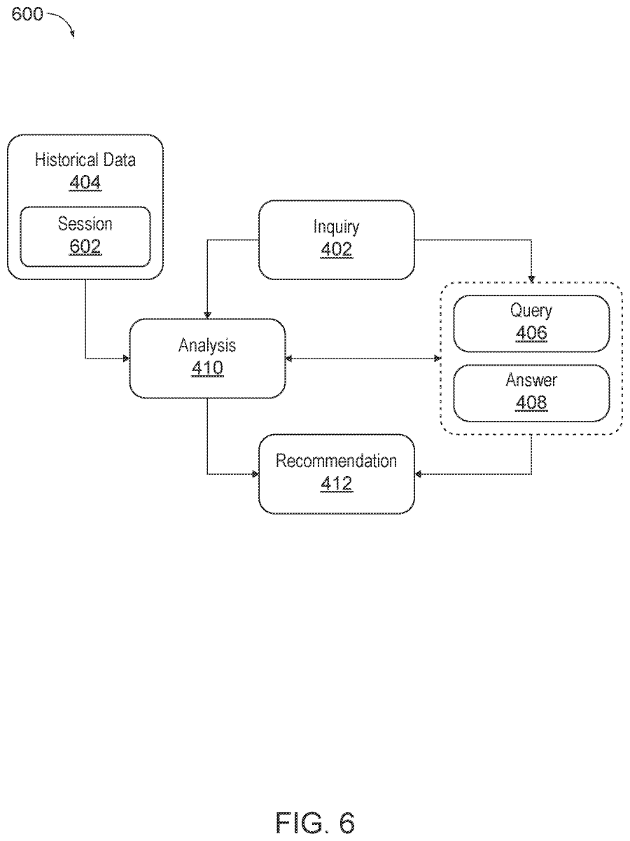

In one embodiment, the thermometer 102 is an infrared thermometer capable of measuring temperatures from a first anatomical region, such as the inner ear, and a second anatomical region, such as the temple of the subject. The profile may include the historical data 404 organized into sets. For example, the sets may include a first set of historical data for temperature measurements sensed via the inner ear, and a second set of the historical data 404 for temperature measurements sensed via the temple. Accordingly, the sets of the historical data 404 may be associated with anatomical regions. As another example, suppose that the temperature module 120 receives a first previous measurement from a first device, such as the thermometer 102, and a second previous measurement from a second device, such as the alternative thermometer. The profile may include a first set of the historical data 404 for temperature measurements received from the first device, and a second set of the historical data 404 for temperature measurements received from the second device. Accordingly, the profile of the subject may include the temperature measurements of the subject as well as characteristics about the temperature measurements. Therefore, the sets of the historical data 404 may be associated with profiles, devices, sessions as will be described with respect to FIG. 6, among other characteristics.

The temperature module 120 may generate the inconsistency notification 510 based on the characteristics of the historical data 404 including the metadata. For example, temperature measurements taken from one anatomical region may appear inconsistent when compared with the temperature measurements taken from a second anatomical region. Thus, the temperature module 120 may provide the inconsistency notification 510 to the user 204 when temperature measurements sharing a first characteristic vary a predetermined amount.

At block 304, the method 300 includes the analysis module 122 generating a query 406 for the user based on the inquiry 402 and the current measured temperature. The query 406 allows the analysis module 122 to receive more targeted information about the inquiry 402 from the user 204. The query 406 may be directed to the user 204, the subject, the current measured temperature, the profile of the subject and/or the user 204, or the location of the subject and/or the user 204. At block 306, the method 300 includes the analysis module 122 receiving an answer 408 from the user 204 in response to the query 406. The answer 408 may be may input by the user 204 in a similar manner as the inquiry 402, for example, as a free word input in which the user provides keywords. In another embodiment, the analysis module 122 may provide the user 204 a plurality of answers from which the user 204 can select. The analysis module 122 receives the selection from the user 204 as the answer 408. Accordingly, the user 204 can participate in a query and answer session based on the inquiry 402.

Returning to the block 304, in one embodiment, the queries provided by the analysis module 122 may be based on the inquiry category of the set of inquiries 502. For example, suppose that the user 204 selected the unexpected reading category 504 from the set of inquiries 502. The query 406 may be defined to determine whether the current measured temperature was unexpected because it was low or high. For example, with respect to block 304, the user 204 may be presented with graphics to select from on the display 500 that define the current measured temperature as too low or too high. Suppose the query 406 states “Was the current measured temperature too high?” The answer 408 may be received as “yes.” In this manner, the query 406 may be provided to the user 204 as a selectable choice. Accordingly, at block 306, the analysis module 122 receives the answer 408 from the user 204 in response to the query 406 by the user 204 selecting one of the graphics associated with the current measured temperature being either too low or too high. In this manner, the answer 408 may be directly related to the inquiry 402 made by the user 204.

As another example, suppose that the user 204 selects the readings vary category 506. The query 406 associated with the readings vary category 506 may be defined to determine if a current temperature measurement is trending with previous temperature measurements or deviating from the previous temperature measurements. For example, with respect to block 304, the user 204 may be presented with graphics to select from on the display 500 that define the current measured temperature as following a pattern or being anomalous. Accordingly, at block 306, the analysis module 122 receives the answer 408 from the user 204 in response to the query 406 by selecting a pattern or anomaly. Accordingly, the query 406 allows the analysis module 122 to gather more information about the experience of the user 204.

The query 406 also allows the analysis module 122 to request information that is unavailable to the computing device 104. Returning to the example from above, suppose that the user 204 measures his or her temperature using an alternative thermometer (not shown) and receives a previously measured temperature. Next the user 204 receives the current measured temperature. With respect to block 304, the query 406 may request access to temperature data from the alternative thermometer. Accordingly, at block 306, the analysis module 122 receives the answer 408 from the user 204 in response to the query 406 as access or denial to the temperature data from the alternative thermometer. Thus, the query 406 may also allow the analysis module to gather more information about the circumstances of the alternative thermometer, the thermometer 102, the user 204, the subject, or the environment of the user 204 or the subject. In another embodiment, the query 406 may include a request that the user specify a set of historical data. For example, the query 406 may include a request to the user to specify whether the current measured temperature was measured at the first anatomical region or the second anatomical region.

At block 308, the method 300 includes the analysis module 122 analyzing the current measured temperature in an analysis 410 of the historical data 404. The analysis 410 correlates the current measured temperature with the historical data 404. For example, the analysis 410 may be a trend line analysis to determine if the measured temperatures of the subject are trending upward or downward. In some embodiments, the analysis 410 may include determining a variability value for the historical data, for example, based on a trendline. The analysis 410 may further include generating the query 406 to confirm the profile of the user in response to variability value exceeding a variability threshold. The analysis 410 may further include the analysis module 122 analyzing the current measured temperature in the analysis 410 based on the profile. The profile may also include location data (e.g., location coordinates, ambient environmental temperature, the local temperature, case rates of disease or infection, etc.) and/or demographic information about the user. The demographic data may include characteristics such as age, race, ethnicity, employment status, education level, income, and address among others.

In some embodiments, the analysis module 122 may include receiving or identifying physiological data. Physiological data may include heart information, such as, heart rate, heart rate pattern, blood pressure, oxygen content, among others. Physiological data can also include brain information, such as, electroencephalogram (EEG) measurements, functional near infrared spectroscopy (fNIRS), functional magnetic resonance imaging (fMRI), among others. Physiological data can also include digestion information, respiration rate information, salivation information, perspiration information, pupil dilation information, body temperature, muscle strain, as well as other kinds of information related to the autonomic nervous system or other biological systems of the vehicle occupant. Physiological data may further include recognition data (e.g., biometric identification) used to identify the user 204. For example, recognition data can include a pre-determined heart rate pattern associated with the user 204, among other types of recognition data. The profile of the user 204 may be identified based on the recognition data.

The analysis module 122 may receive the physiological data as sensor data 110 from the thermometer 102. The recognition data and other types of physiological data may additionally or alternatively be stored at various locations (e.g., the memory 114, the data store 116, a memory integrated with the wearable computing devices, a remote database) and accessed by the analysis module 122.

The analysis 410 of the analysis module 122 may further identify anomalous temperature measurements using for example a least squares regression tool. The analysis 410 may be based on predictive analytics. For example, the analysis may predict a predicted measured temperature based on the historical data 404, the query 406, and the answer 408 and compare the predicted measured temperature to the current measured temperature. Accordingly, the analysis 410 may utilize the query 406 and the answer 408.

The analysis module may perform steps in parallel. For example, turning to FIG. 4, when an inquiry 402 is received by the analysis module 122, the analysis module 122 generates the query 406 based on the inquiry 402 and receives the answer 408 from the user 204 in response to the query 406, as described with respect to blocks 304 and 306 respectively. In this embodiment, the analysis 410 of the current measured temperature based on the historical data 404, as described with respect with block 308, may happen in parallel with generating the query and receiving the answer 408, to generate a recommendation 412.

The recommendation 412 is generated by the recommendation module 124 at block 310 of the method 300. The recommendation 412 is associated with the inquiry 402 based on the answer 408 and the analysis 410. The recommendation 412 may indicate a confidence level in the current measured reading. For example, the recommendation 412 may indicate that the recommendation module 124 is confident, somewhat confident, or not confident in the current measured reading based on the answer 408 and the analysis 410.

The recommendation 412 may also provide suggestions (e.g., tips for operation, information about the thermometer 102, etc.). The suggestions may assist the user 204 in troubleshooting the current measured temperature based on the inquiry 402. For example, the recommendation 412 may include adjustments that can be made to the thermometer 102 or the portable device 130. Adjustments may be based on the execution module 126. In one embodiment, the recommendation 412 may be a notification displayed on the display 500 that includes alignment instructions for the thermometer 102. The recommendation 412 may be text, audio, image, video, or projection, among others. For example, the recommendation 412 may be a video that illustrates how the thermometer 102 should be aligned relative to the subject. As one example, the recommendation 412 may be further based on the first set of the historical data or the second set of the historical data. For example, the query may request that the user specify whether the current measured temperature is from a first anatomical region, such as the temple, or a second anatomical region, such as the inner ear. The recommendation 412 for how to manipulate the device maybe based on answer 408. For example, the recommendation may be to take a measurement from a different anatomical region or how to align the device relative to the answered anatomical region.

The recommendation 412 may additionally or alternatively indicate reasons or an issue associated with an inquiry category when the inquiry 402 is based on the set of inquiries 502. Suppose the readings vary category 506 is selected by the user 204. The recommendation 412 may give a possible reason for the various readings. For example, suppose that the thermometer 102 is an ear thermometer, the recommendation 412 may state that alignment down the ear canal can be inconsistent causing the readings to vary.

The recommendation 412 may also include information about the user 204 and/or subject. For example, suppose the user 204 indicated that the current measured temperature was in the unexpected reading category 504. The query 406 and the answer 408 may question the user 204 about the actions, environment, and status of the subject. As one example, the query 406 may question the recent activity of the subject. Suppose that the subject has just finished a cardiovascular workout. The answer 408 may indicate a current heartrate of the subject, the activity level of the cardiovascular workout, or type of activity, among others.

The recommendation 412 may then indicate that the current measured temperature is not anomalous but due to the recent activity of the subject. The recommendation 412 may further advise a future measurement be taken. For example, the recommendation 412 may include that a next reading be taken in a predetermined amount of time, such as 20 minutes, to give the subject adequate time to cool down before taking the next reading.

In this manner, the recommendation 412 offers support to the user 204 when the user 204 is unsure of the current temperature measurement. Based on the recommendation 412, the user 204 may have increased confidence in the measured temperature from the thermometer 102. Because the recommendation 412 is based on the historical data 404 and the answer 408 from the user 204, the user 204 is provided with relevant, prioritized, and personalized guidance.

Although previous examples include one query and one answer, the analysis module 122 may generate additional queries and receive additional answers based on the additional queries. For example, FIG. 6 illustrates one embodiment based on the inquiry 402 in the readings vary category 506. Here, the analysis 410 may be based on session limiting. For example, the historical data 404 may include hundreds of readings of multiple subjects. To ensure the analysis 410 is relevant to the current measured temperature, the analysis 410 may be performed on previously measured temperatures that are a subset of the historical data 404. For example, a session 602 may include a subset of the historical data corresponding to ten minutes prior to the current measured temperature. By limiting the historical data 404 to the previously measured temperatures based on the session 602, data from previous sessions is excluded from the analysis 410.

The session 602 may be defined by a predetermined amount of time that precedes the current measured temperature or timeline including the current measured temperature. As another example, the session 602 may be defined by a cluster of previously measured temperatures in the historical data 404. For example, a thermometer 102 may be used sporadically such that the historical data 404 associated with the thermometer 102 is time dependent based on use. In some embodiments, the analysis module 122 may analyze the historical data 404 to identify clusters. The session 602 may be limited to a cluster of previously measured temperatures that includes the current measured temperature.

In another embodiment, the session 602 may be based on a profile of a subject or a user 204. For example, the query 406 may request the user 204 verify the profile is associated with the user 204. The query 406 may also request the user 204 verify that the profile is associated with a subject. In this manner, the query 406 may request verification of the profile. The current measured temperature may then be associated with the profile such that the historical data 404 is associated with specific subjects or users. The session 602 may be limited to temperature measurements associated with those specific subjects or users for a predetermined amount of time or time dependent cluster.

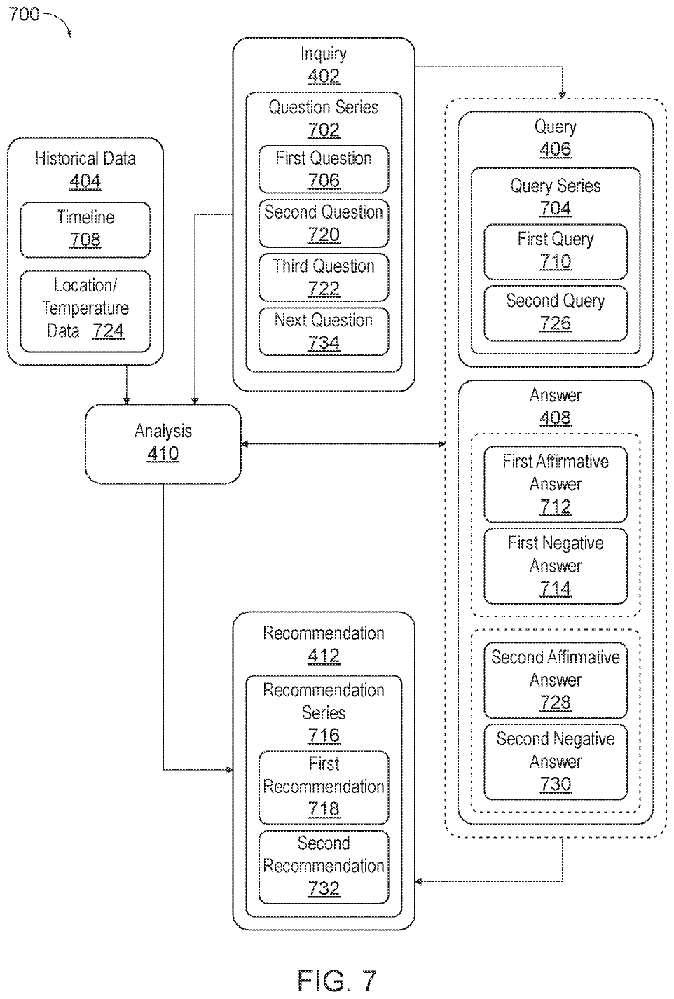

Although the examples above describe a single query and answer, the query and answer session may include any number of queries. Turning to FIG. 7, when an inquiry is received a question series 702 may be generated by the analysis module 122. The question series 702 includes a number of questions that identify information needed to respond to the inquiry 402. For example, one or more questions of the question series 702 may identify information needed to perform the analysis 410. To receive the information identified in the questions of the question series 702, the analysis module 122 generates a query series 704 including one or more queries and answers, such as the query 406 and receiving the answer 408.

For example, suppose the first question 706 of the question series 702 is attempting to determine whether there are previously measured temperatures below 98.6° F. The analysis module 122 may access a timeline 708 and the historical data 404 to resolve the first question 706. Based on the resolution, the query series 704 may include a first query 710. Here, the first query 710 is directed to whether the user 204 was expecting a normal reading of 98.6° F. In response to the first query 710, the analysis module 122 may receive a first affirmative answer 712 or a first negative answer 714.

If the first affirmative answer 712 is received by the analysis module 122 in response to the first query 710, the recommendation module 124 generates a recommendation 412 of the recommendation series 716, such as the first recommendation 718. If the first negative answer 714 is received by the analysis module 122 in response to the first query 710, the analysis module 122 moves to the second question 720 of the question series 702. The second question 720 may cause the analysis module 122 to perform another round of analysis using the historical data 404. For example, the analysis module 122 may access the timeline 708 and perform analysis with respect to the second question 720. Therefore, in addition to using multiple queries, the analysis module 122 may perform multiple rounds of analysis.

Suppose that the analysis continues to a third question 722 of the question series 702. The third question 722 may access other types of historical data 404 or the outside temperature based on the profile. For example, the analysis module 122 may include location temperature data 724 for the subject or the user 204. Based on the resolution of the third question 722, the query series 704 may include a second query 726. Here, the second query 726 is directed to whether the user 204 and/or subject has come in from a cold environment. In response to the second query 726, the analysis module 122 may receive a second affirmative answer 728 or a second negative answer 730. If the second affirmative answer 728 is received by the analysis module 122 in response to the second query 726, the recommendation module 124 generates the second recommendation 732 of the recommendation series 716.

If the second negative answer 730 is received by the analysis module 122 response to the second query 726, the analysis module 122 moves to a next question 734 of the question series 702. In this manner, the query 406, the answer 408, and the analysis 410 of the analysis module 122 may be performed iteratively until the recommendation 412 is generated.

Another example of the method 700 may include the user 204 receiving a first forehead temperature of 96.8° F., a second temperature of 97.3° F., and a third temperature of 98.1° F. The user may be concerned because the temperature is not consistent and may input “readings vary” 506 by touching a screen of the thermometer 102 or portable device 130. One potential reason for this is that the subject has come in from a cold environment outside and is gradually warming up and that the subject's forehead does not properly reflect their body temperature. The analysis module 122 may determine the local temperature of the user 204 based on the location data. Based on the local temperature, the analysis 410 may include determining that the local temperature is a threshold amount colder than a standard room temperature. Accordingly, the query 406 may query the user 204 whether the subject has come in from the outside. If the user 204 provides an affirmative answer 712, then the first recommendation 718 may be to allow the subject to wait a predetermined amount of time to acclimate to the ambient temperature of the thermometer 102. If instead, the user 204 provides a negative answer 714, the analysis module 122 may generate a second query 726 to determine whether the thermometer 102 has been in a different ambient environment.

Another example of the method 700 may include a user 204 receiving a first ear temperature of 98.8° F., a second ear temperature of 98.3° F., and a third temperature of 98.7° F. The user 204 may be concerned because the temperature is not consistent and may input “readings vary” 506 by touching a screen of the thermometer 102 or portable device 130. The first query 710 may request information about the placement of the thermometer 102, such as if the user 204 is inserting a probe of the thermometer 102 properly. If the user 204 provides an affirmative answer 712, the second query 726 may ask whether the user 204 has switched ears from left to right. Because ears are known to vary in temperature from one to the other, this may be a possible explanation. Accordingly, the recommendation 412 may provide the user 204 with instructions to use only the left ear, or only the right ear for consistency.

As discussed, the type of questions and the order in which they are presented can change based on both previous readings, the one more answers 408 from the user 204 in response to the one or more queries 406 and/or other parameters (e.g., exterior temperature, time of year, pulse of the subject, blood pressure of the subject, etc.). Furthermore, the type of queries and the sequence of the questions of the question series 702 and the resulting recommendations 412 may be influenced by a statistical analysis of the historical queries made, not only by a particular device, but by analyzing queries across many devices (e.g., thermometer 102, computing device 104, etc.) in many locations. This analysis 410 can be further enhanced by employing machine learning and/or AI algorithms of a large data set, so that the sequence of the question series 702 can become more efficient over time, enabling a user 204 to quickly get to a trusted reading.

After the recommendation 412 is given, the computing device 104 may receive a confirmation response from the user. The confirmation response may be an option displayed on the screen of the thermometer 102 or portable device 130 that allows the user 204 to indicate that the user 204 trusts the temperature and that their doubt is resolved. Depending on the inquiries 402 from the user 204, the queries 406, the recommendation 412, and the confirmation response that the user's issue is resolved, the computing device 104 can remember and preferentially prioritize that sequence of questions in the question series 702 to resolve problems more quickly in the future. In this manner, the computing device 104 may learn from previous interactions with users and subjects across multiple thermometers. For example, the sequence of questions of the question series 702 may be based how quickly previous inquiries of previous interactions were resolved. Accordingly, the query 406 and the recommendations is affected by an analysis of historical data 404, answer 408, and confirmation responses from user 204

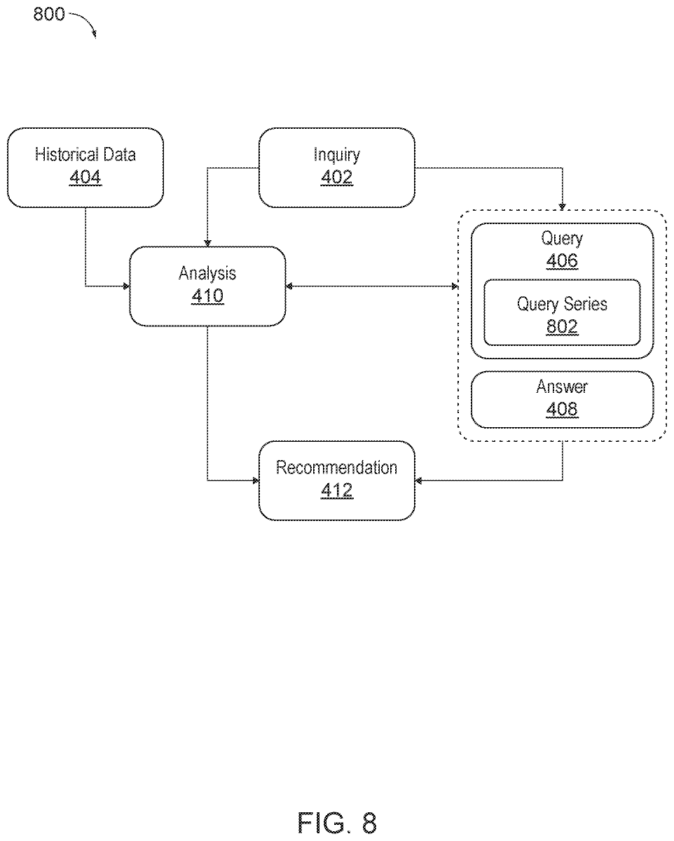

FIG. 8 is an exemplary process flow for increasing thermometric reliability according to one aspect. In the exemplary process flow of the method 800, a query 802 may include a number of queries. For example, the query 802 may request a number of items of health information, such as: is the ear covered, is there an ear infection, or have you taken a fever reducing medicine. The query 802 may also request information about the subject and/or user 204 such as: recent activity, taken a shower, had a workout, etc. Suppose the query 802 is displayed on the display 500 of the portable device 130. The query 802 may be displayed as a group of radio buttons selectable by the user 204. In response to selecting a radio button associated with a request of the query 802, a corresponding recommendation is generated by the recommendation module 124.

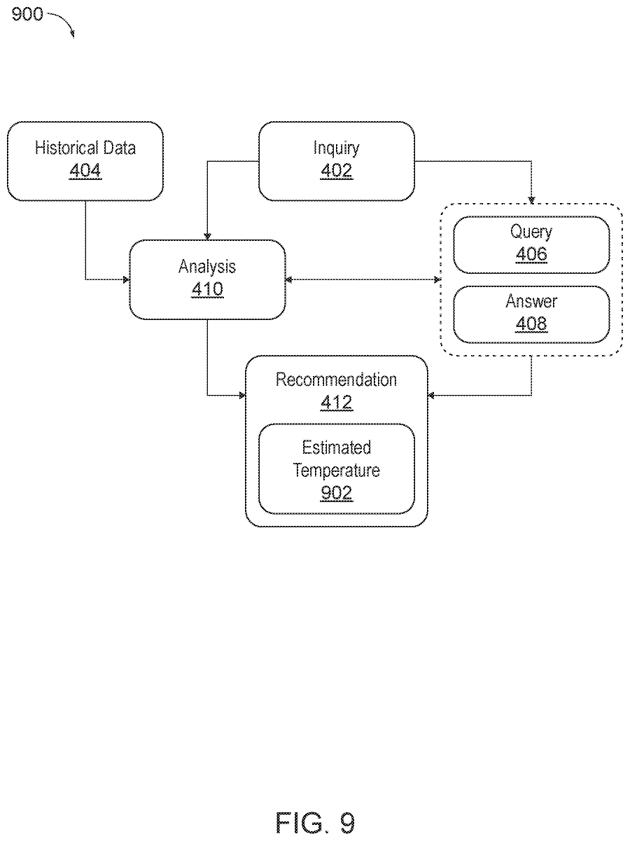

FIG. 9 is another exemplary process flow for increasing thermometric reliability, according to one aspect. In some embodiments, the recommendation 412 may include an estimated temperature based on the answer 408. For example, the temperature module 120 may receive an inquiry in the different result category 508 based on the user 204 using multiple devices to sense the current measured temperature, as discussed above. Suppose that the current measured temperature may be received from the thermometer 102 which is an ear sensing thermometer. The analysis module 122, may request information about the alternative thermometer such as the make, model, or type (e.g., forehead, under arm, ear, rectal, oral, etc.), among others. Based on the answer 408 given by the user 204, a recommendation 412 is generated. Further suppose that the user 204 answers that the alternative thermometer is an oral thermometer. The corresponding recommendation may include an estimated temperature 902 that converts the current measured temperature to an estimated equivalent of an oral temperature.

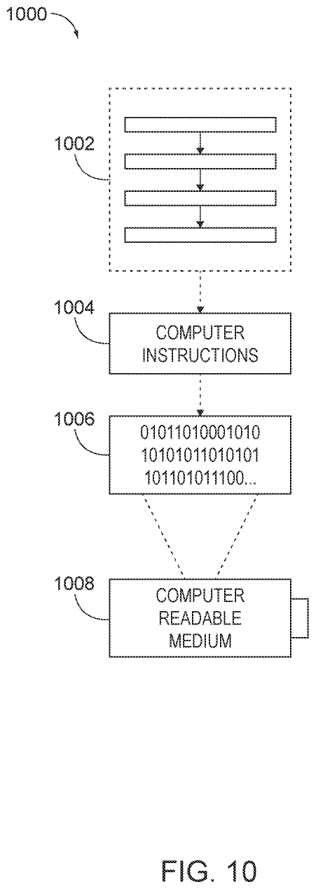

Still another aspect involves a computer-readable medium including processor-executable instructions configured to implement one aspect of the techniques presented herein. An aspect of a computer-readable medium or a computer-readable device devised in these ways is illustrated in FIG. 10, wherein an implementation 1000 includes a computer-readable medium 1008, such as a CD-R, DVD-R, flash drive, a platter of a hard disk drive, etc., on which is encoded computer-readable data 1006. This encoded computer-readable data 1006, such as binary data including a plurality of zero's and one's as shown in 1006, in turn includes a set of processor-executable computer instructions 1004 configured to operate according to one or more of the principles set forth herein. In this implementation 1000, the processor-executable computer instructions 1004 may be configured to perform a method 1002, such as the method 300 of FIG. 3, the method 600 of FIG. 6, the method 700 of FIG. 7, the method 800 of FIG. 8, and/or the method 900 of FIG. 9. In another aspect, the processor-executable computer instructions 1004 may be configured to implement a system, such as the operating environment 100 of FIG. 1. Many such computer-readable media may be devised by those of ordinary skill in the art that are configured to operate in accordance with the techniques presented herein.

As used in this application, the terms “component”, “module,” “system”, “interface”, and the like are generally intended to refer to a computer-related entity, either hardware, a combination of hardware and software, software, or software in execution. For example, a component may be, but is not limited to being, a process running on a processor, a processing unit, an object, an executable, a thread of execution, a program, or a computer. By way of illustration, both an application running on a controller and the controller may be a component. One or more components residing within a process or thread of execution and a component may be localized on one computer or distributed between two or more computers.

Further, the claimed subject matter is implemented as a method, apparatus, or article of manufacture using standard programming or engineering techniques to produce software, firmware, hardware, or any combination thereof to control a computer to implement the disclosed subject matter. The term “article of manufacture” as used herein is intended to encompass a computer program accessible from any computer-readable device, carrier, or media. Of course, many modifications may be made to this configuration without departing from the scope or spirit of the claimed subject matter.

The term “computer readable media” includes communication media. Communication media typically embodies computer readable instructions or other data in a “modulated data signal” such as a carrier wave or other transport mechanism and includes any information delivery media. The term “modulated data signal” includes a signal that has one or more of its characteristics set or changed in such a manner as to encode information in the signal.

Although the subject matter has been described in language specific to structural features or methodological acts, it is to be understood that the subject matter of the appended claims is not necessarily limited to the specific features or acts described above. Rather, the specific features and acts described above are disclosed as example aspects. Various operations of aspects are provided herein. The order in which one or more or all of the operations are described should not be construed as to imply that these operations are necessarily order dependent. Alternative ordering will be appreciated based on this description. Further, not all operations may necessarily be present in each aspect provided herein.

As used in this application, “or” is intended to mean an inclusive “or” rather than an exclusive “or”. Further, an inclusive “or” may include any combination thereof (e.g., A, B, or any combination thereof). In addition, “a” and “an” as used in this application are generally construed to mean “one or more” unless specified otherwise or clear from context to be directed to a singular form. Additionally, at least one of A and B and/or the like generally means A or B or both A and B. Further, to the extent that “includes”, “having”, “has”, “with”, or variants thereof are used in either the detailed description or the claims, such terms are intended to be inclusive in a manner similar to the term “comprising”.

Further, unless specified otherwise, “first”, “second”, or the like are not intended to imply a temporal aspect, a spatial aspect, an ordering, etc. Rather, such terms are merely used as identifiers, names, etc. for features, elements, items, etc. For example, a first channel and a second channel generally correspond to channel A and channel B or two different or two identical channels or the same channel. Additionally, “comprising”, “comprises”, “including”, “includes”, or the like generally means comprising or including, but not limited to.

It will be appreciated that variations of the above-disclosed embodiments and other features and functions, or alternatives or varieties thereof, may be desirably combined with many other different systems or applications. Also, various presently unforeseen or unanticipated alternatives, modifications, variations or improvements therein may be subsequently made by those skilled in the art which are also intended to be encompassed by the following claims.

1. A system for increasing thermometric reliability, the system comprising:
a temperature module configured to receive an inquiry from a user about a current measured temperature of a subject, wherein the current measured temperature is measured by a thermometer, and wherein the inquiry indicates that the user doubts the current measured temperature;
an analysis module configured to: generate a query based on the inquiry and the current measured temperature; receive an answer in response to the query; and analyze the current measured temperature in an analysis of historical data; and a recommendation module configured to generate a recommendation associated with the inquiry based on the answer and the analysis.

2. The system of claim 1, wherein the historical data includes previously measured temperatures for the subject that precede the current measured temperature by a predetermined amount of time that defines a session, wherein the previously measured temperatures for the subject are associated with a profile for the subject.

3. The system of claim 2, wherein the analysis is a trend line analysis of the historical data and the current measured temperature.

4. The system of claim 2, wherein the analysis includes:
determining a variability value for the historical data; and
generating the query to confirm the profile of the subject in response to variability value exceeding a variability threshold.

5. The system of claim 4, wherein the profile includes location data associated with the subject, or demographic information about the subject or historical information about the subject's temperature.

6. The system of claim 4, wherein the query requests verification of the profile.

7. The system of claim 4, wherein the profile includes a first set of the historical data associated with a first anatomical region and a second set of the historical data associated with a second anatomical region different than the first anatomical region.

8. The system of claim 7, wherein the query requests the user specify whether the current measured temperature was measured at the first anatomical region or the second anatomical region.

9. The system of claim 1, wherein the recommendation includes manipulating the thermometer.

10. A method for increasing thermometric reliability, the method comprising:
receiving an inquiry from a user about a current measured temperature of a subject, wherein the current measured temperature is measured by a thermometer, and wherein the inquiry indicates that the user doubts the current measured temperature;
generating a query based on the inquiry and the current measured temperature;
receiving an answer in response to the query;
analyzing the current measured temperature in an analysis of historical data; and
generating a recommendation associated with the inquiry based on the answer and the analysis.

11. The method of claim 10, wherein the historical data includes previously measured temperatures for the user that precede the current measured temperature by a predetermined amount of time that defines a session, wherein the previously measured temperatures for the subject are associated with a profile for the subject.

12. The method of claim 11, wherein the analysis is a trend line analysis of the historical data and the current measured temperature.

13. The method of claim 11, wherein the analysis includes:
determining a variability value for the historical data; and
generating the query to confirm the profile of the user in response to variability value exceeding a variability threshold.

14. The method of claim 13, wherein the profile includes location data associated with the subject, and demographic information about the subject.

15. The method of claim 13, wherein the profile includes physiological data associated with the subject.

16. The method of claim 10, further comprising:
receiving a confirmation response from the user that the inquiry is resolved.

17. The method of claim 16, wherein the query includes a question series having a plurality of questions, and wherein a sequence of questions of the plurality questions is based the confirmation response of a previous interaction.

18. A non-transitory computer readable storage medium storing instructions that when executed by a computer having a processor to perform a method, the method comprising:
receiving an inquiry from a user about a current measured temperature, wherein the current measured temperature is measured by a thermometer, and wherein the inquiry indicates that the user doubts the current measured temperature;
generating a query based on the inquiry and the current measured temperature;
receiving an answer in response to the query;
analyzing the current measured temperature in an analysis of historical data; and
generating a recommendation associated with the inquiry based on the answer and the analysis.
